(19) United States
(12) Patent Application Publication
(10) Pub. No.: US 2010/0234310 A1
(43) Pub. Date: Sep. 16, 2010

(54) ESTROGENIC COMPOUNDS AND THEIR METHODS OF USE

(75) Inventor: Isaac Cohen, Piedmont, CA (US)

Correspondence Address: WILSON, SONSINI, GOODRICH & ROSATI, 650 PAGE MILL ROAD, PALO ALTO, CA, 94304-1050, US

(73) Assignee: Bionovo, Inc., Emeryville, CA (US)

(21) Appl. No.: 12/712,980

(22) Filed: Feb. 25, 2010

Related U.S. Application Data
(60) Provisional application No. 61/159,346, filed on Mar. 11, 2009.

(51) Int. Cl. A61K 31/7048; A61K 31/353; A61P 35/00

(52) U.S. Cl. 514/27; 514/456

(57) ABSTRACT

Isolated compounds and combinations of isolated compounds isolated from Epimedium grandiflorum Morr. are estrogenic, and can be used to treat one or more symptoms of menopause and/or ER-positive cancer. The compounds and combinations may be prepared as pharmaceutical compositions for administration to mammals, such as humans, for the treatment of solid cancers, such as epithelial cancers. Such epithelial cancers include breast cancer, uterine cancer, cervical cancer, ovarian cancer, vulvar cancer, endometrial cancer.

### CROSS-REFERENCE AND PRIORITY CLAIM

This application claims priority under 35 U.S.C. §119(e) to U.S. provisional application 61/159,346, filed Mar. 11, 2009, which is incorporated herein by reference in its entirety.

### BACKGROUND OF THE INVENTION

Hormone replacement therapy (HRT) has been used successfully to treat a variety of conditions, such as osteoporosis, increased risk of cardiovascular disease in post-menopausal women and climacteric symptoms, such as hot flashes, decreased libido and depression. However, HRT with estradiol (E₂), either alone or in combination with progestin, can lead to undesirable effects. In fact, a recent Women's Health Initiative (WHI) study was abruptly halted when preliminary results showed that HRT was associated with a 35% increased risk of breast cancer.

Breast cancer can be treated or prevented by using a so-called selective estrogen receptor modulator (SERM), such as tamoxifen. (Before the approval of tamoxifen, breast cancer treatment of pre-menopausal women often included removing the ovaries in order to reduce the cancer-stimulating effect of estrogen.) Tamoxifen appears to selectively block the cancer-inducing effects of estrogen in breast tissues of pre-menopausal women. Another SERM, raloxifene, has been approved for treatment of osteoporosis as an alternative to estrogen replacement. In addition to selectively inducing estrogenic effects in bone tissue, long-term administration of raloxifene was also shown to be associated with reduction in the rate of breast cancer in the Multiple Outcomes of Raloxifene Evaluation (MORE) study.

While SERMs such as tamoxifen and raloxifene provide selective reduction in estrogen's cancer-inducing effects in the breast, they are not without their risks. For example both tamoxifen and raloxifene therapy have been associated with increased incidence of hot flushes; and tamoxifen therapy has been shown to increase the risk of uterine (endometrial) cancer. There is thus a need for additional options for the treatment and prevention of estrogenic cancers, such as breast, ovarian, uterine and cervical cancers. In addition, there is a need for additional options for the treatment of symptoms of menopause, such as hot flashes.

These and other needs are met by embodiments of the invention.

### SUMMARY OF THE INVENTION

It has been surprisingly found that certain compounds isolated from the species Epimedium grandiflorum Morr. possess estrogenic activity. In particular, it has been found that E. grandiflorum Morr. contains Apigenin, Luteolin, Kaempferol, Narigenin, Quercetin, Quercetrin, and Quercetin-3-O-β-glucopyranoside, which have been found to possess estrogenic activity, and in particular ER-β-mediated estrogenic activity. It has further been found that combinations of certain of these compounds possess not only estrogenic activity greater than would have been predicted from their individual in vitro activities, but also greater activity against certain types of cancer than would have been predicted from their individual in vitro activities. It is thus an aspect of this invention to employ one or more, two or more, three or more, four or more, five or more, six or all seven of Apigenin, Luteolin, Kaempferol, Narigenin, Quercetin, Quercetrin, and Quercetin-3-O-β-glucopyranoside in the treatment of one or more symptoms of menopause. In some embodiments, the pharmaceutical composition comprises Apigenin and/or Luteolin, and further optionally comprises one or more of Kaempferol, Narigenin, Quercetin, Quercetrin, and Quercetin-3-O-β-glucopyranoside, with the proviso that the pharmaceutical composition does not contain any one of the following: Scutellarein, Isoscutellarein, Carthamidin, and Isocarthamidin. It is further an aspect of this invention to employ one or more, two or more, three or more, four or more, five or more, six or all seven of Apigenin, Luteolin, Kaempferol, Narigenin, Quercetin, Quercetrin, and Quercetin-3-O-β-glucopyranoside in the treatment of cancer, and in particular breast cancer, uterine cancer, ovarian cancer, cervical cancer, vulvar cancer, vaginal cancer, endometrial cancer, or a metastatic cancer originating in the breast, uterus, ovary, cervix, vulva, vagina, or endometrium. In another aspect of the invention, there are provided pharmaceutical compositions comprising an optional pharmaceutically acceptable excipient and one or more, two or more, three or more, four or more, five or more, six or all seven of Apigenin, Luteolin, Kaempferol, Narigenin, Quercetin, Quercetrin, and Quercetin-3-O-β-glucopyranoside. In some embodiments, wherein the composition contains Apigenin, Luteolin or a combination thereof, the composition further contains Kaempferol, Narigenin, Quercetin, Quercetrin, or Quercetin-3-O-β-glucopyranoside, or a combination of two or more thereof.

Some embodiments described herein provide a pharmaceutical composition comprising a pharmaceutically acceptable excipient and one or more members of the group consisting of Apigenin, Luteolin, Kaempferol, Narigenin, Quercetin, Quercetrin, and Quercetin-3-O-β-glucopyranoside. In some embodiments, the pharmaceutical comprises two or more members of the group consisting of Apigenin, Luteolin, Kaempferol, Narigenin, Quercetin, Quercetrin, and Quercetin-3-O-β-glucopyranoside. In some embodiments, the pharmaceutical composition comprises three or more members of the group consisting of Apigenin, Luteolin, Kaempferol, Narigenin, Quercetin, Quercetrin, and Quercetin-3-O-β-glucopyranoside. In some embodiments, the pharmaceutical composition comprises four, five, six, or seven of the members of the group consisting of Apigenin, Luteolin, Kaempferol, Narigenin, Quercetin, Quercetrin, and Quercetin-3-O-β-glucopyranoside.

Some embodiments described herein provide a method of selectively inducing apoptosis in a population of hyperproliferative cells in a mammal, comprising administering to the mammal a therapeutically effective amount of one of the a pharmaceutical composition comprising one or more, two or more, three or more, four or more, five or more, six or all seven of Apigenin, Luteolin, Kaempferol, Narigenin, Quercetin, Quercetrin, and Quercetin-3-O-β-glucopyranoside. Some embodiments described herein provide a method of selectively inducing apoptosis in a cancer in a mammal, comprising administering to the mammal a therapeutically effective amount of one of the foregoing compositions. Some embodiments described herein provide a method of treating cancer in a mammal, comprising administering to the mammal a therapeutically effective amount of one of the foregoing compositions. Some embodiments described herein provide a method of treating breast, ovarian, uterine, vaginal, cervical, vulvar, or endometrial cancer in a mammal, comprising administering to the mammal a therapeutically effective amount of one of the foregoing compositions. Some embodiments described herein provide a method of treating metastatic breast, ovarian, uterine, vaginal, cervical, vulvar, or endometrial cancer in a mammal, comprising administering to the mammal a therapeutically effective amount of one of the foregoing compositions. In some embodiments, the treated mammal is a human. Some embodiments described herein also provide a use of a composition described above for preparation of a medicament for selectively inducing apoptosis in a population of hyperproliferative cells in a mammal. Some embodiments provide a use of one of the above-described compositions for preparation of a medicament for selectively inducing apoptosis in a cancer in a mammal. Some embodiments described herein also provide a use of one of the above-described compositions of for preparation of a medicament for treating cancer in a mammal. Some embodiments described herein provide a use of a composition described above for preparation of a medicament for treating breast cancer, uterine cancer, ovarian cancer, cervical cancer, vulvar cancer, vaginal cancer, or endometrial cancer in a mammal. In some embodiments, there is provided a use of a composition described above for preparation of a medicament for treating metastatic cancer originating in metastatic cancer originating in the breast, uterus, ovary, cervix, vulva, vagina, or endometrium in a mammal. In some embodiments, the treated mammal is a human.

Some embodiments described herein provide a pharmaceutical composition, which consists essentially of an optional pharmaceutically acceptable excipient and one or more, two or more, three or more, four or more, five or more, six or all seven members of the group consisting of Apigenin, Luteolin, Kaempferol, Narigenin, Quercetin, Quercetrin, and Quercetin-3-O-β-glucopyranoside. In some embodiments, the pharmaceutical composition consists of a pharmaceutically acceptable excipient and one or more, two or more, three or more, four or more, five or more, six or all seven members of the group consisting of Apigenin, Luteolin, Kaempferol, Narigenin, Quercetin, Quercetrin, and Quercetin-3-O-β-glucopyranoside. Some embodiments described herein provide a method of selectively inducing apoptosis in a population of hyperproliferative cells in a mammal, comprising administering to the mammal a therapeutically effective amount of a pharmaceutical composition consisting of an optional pharmaceutically acceptable excipient and one or more, two or more, three or more, four or more, five or more, six or all seven of the group consisting of Apigenin, Luteolin, Kaempferol, Narigenin, Quercetin, Quercetrin, and Quercetin-3-O-β-glucopyranoside. Some embodiments described herein provide a method of selectively inducing apoptosis in a cancer in a mammal, comprising administering to the mammal a therapeutically effective amount of one of the foregoing compositions. Some embodiments described herein provide a method of treating cancer in a mammal, comprising administering to the mammal a therapeutically effective amount of one of the foregoing compositions. Some embodiments described herein provide a method of treating breast cancer, uterine cancer, ovarian cancer, cervical cancer, vulvar cancer, vaginal cancer, or endometrial cancer in a mammal, comprising administering to the mammal a therapeutically effective amount of one of the foregoing compositions. Some embodiments described herein provide a method of treating metastatic cancer originating in the breast, uterus, ovary, cervix, vulva, vagina, or endometrium in a mammal, comprising administering to the mammal a therapeutically effective amount of one of the foregoing compositions. In some embodiments, the treated mammal is a human. Some embodiments described herein also provide a use of a composition described above for preparation of a medicament for selectively inducing apoptosis in a population of hyperproliferative cells in a mammal. Some embodiments provide a use of one of the above-described compositions for preparation of a medicament for selectively inducing apoptosis in a cancer in a mammal. Some embodiments described herein also provide a use of one of the above-described compositions of for preparation of a medicament for treating cancer in a mammal. Some embodiments described herein provide a use of a composition described above for preparation of a medicament for treating breast cancer, uterine cancer, ovarian cancer, cervical cancer, vulvar cancer, vaginal cancer, or endometrial cancer in a mammal. In some embodiments, there is provided a use of a composition described above for preparation of a medicament for treating a metastatic cancer originating in the breast, uterus, ovary, cervix, vulva, vagina, or endometrium in a mammal. In some embodiments, the treated mammal is a human.

Other uses and advantages of the present invention will be apparent to the person skilled in the art after having considered the description, including the drawings and claims, herein.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the ER-β agonistic activity of Apigenin, one of several ER-β agonists isolated and identified from Epimedium grandiflorum Morr.

### DETAILED DESCRIPTION OF THE INVENTION

This invention relates to active agents isolated from an extract of Herba Epimedii, and in particular from Epimedium grandiflorum Morr. In some embodiments, a pharmaceutical composition comprising one or more, two or more, three or more, four or more, five or more, six or all seven of Apigenin, Kaempferol, Luteolin, Narigenin, Quercetin, Quercetrin and Quercetin-3-O-glucopyranose, and optionally a pharmaceutically acceptable excipient, has ER-β-specific estrogenic activity in vitro and thus has in vivo activity in the treatment of one or more symptoms of menopause, such as hot flashes, osteoporosis, vaginal atrophy, etc. In some embodiments, a pharmaceutical composition pharmaceutical composition comprising one or more, two or more, three or more, four or more, five or more, six or all seven of Apigenin, Kaempferol, Luteolin, Narigenin, Quercetin, Quercetrin and Quercetin-3-O-β-glucopyranoside, and optionally a pharmaceutically acceptable excipient, has pro-apoptotic activity against one or more cancer cell types, and thus possesses therapeutically beneficial effects against various types of cancer, such as breast cancer, uterine cancer, ovarian cancer, cervical cancer, vulvar cancer, vaginal cancer, or endometrial cancer. In some preferred embodiments, the compositions are free of compounds containing an isoprenyl moiety, such as compounds I, II or III, which were described by Yong et al., U.S. Pub. No. 2003/0170292 A1.

As is described below, certain active compounds and certain combinations of active compounds derived from the herb Epimedium grandiflorum Morr. are active in inhibiting cancer cells.

It has been also been found that certain active species isolated from Epimedium grandiflorum Morr. are active in generation of reactive oxygen species, generation of DNA damage and/or induction of cell death in cancerous cells, in particular breast cancer cells, in vitro. It has also been found that certain unique combinations of active compounds have been found to be particularly active in the induction of generation of reactive oxygen species, DNA damage and/or cell death in cancer cells, and in particular breast cancer cells, in vitro. It is considered therefore that certain isolated compounds, and certain combinations of isolated compounds, are effective in the treatment of cancer, and in particular breast cancer, in mammals, such as humans.

It has been found that Apigenin is active in generation of reactive oxygen species, generation of superoxide, induction of mitochondrial superoxide, induction of cell death, and induction of apoptosis in cancerous cells, in particular breast cancer cells, in vitro. It has been found that Luteolin is active in generation of reactive oxygen species, induction of superoxide, induction of mitochondrial superoxide, induction of cell death and induction of apoptosis in cancerous cells, in particular breast cancer cells, in vitro.

It has been found that a combination of Apigenin and Luteolin is active in generation of reactive oxygen species, generation of superoxide, induction of mitochondrial superoxide, induction of cell death, and induction of apoptosis in cancerous cells, in particular breast cancer cells, in vitro.

In some embodiments, there are provided pharmaceutical compositions comprising one or more pharmaceutically acceptable excipients and one, two, four, five, six, or all seven of Apigenin, Luteolin, Kaempferol, Narigenin, Quercetin, Quercetrin, and Quercetin-3-O-β-glucopyranoside for the treatment of a solid tumor, such as an epithelial cancer, e.g. breast cancer, uterine cancer, ovarian cancer, cervical cancer, vulvar cancer, vaginal cancer, or endometrial cancer. In some embodiments, there are provided pharmaceutical compositions consisting essentially of one or more pharmaceutically acceptable excipients and one, two, four, five, six, or all seven of Apigenin, Luteolin, Kaempferol, Narigenin, Quercetin, Quercetrin, and Quercetin-3-O-β-glucopyranoside for the treatment of a solid tumor, such as an epithelial cancer, e.g. breast cancer, uterine cancer, ovarian cancer, cervical cancer, vulvar cancer, vaginal cancer, or endometrial cancer. In some embodiments, there are provided pharmaceutical compositions consisting of one or more pharmaceutically acceptable excipients and one, two, four, five, six or all seven of Apigenin, Luteolin, Kaempferol, Narigenin, Quercetin, Quercetrin, and Quercetin-3-O-β-glucopyranoside for the treatment of a solid tumor, such as an epithelial cancer, e.g. breast cancer, uterine cancer, ovarian cancer, cervical cancer, vulvar cancer, vaginal cancer, or endometrial cancer.

In some embodiments, there are provided pharmaceutical compositions comprising Apigenin and Luteolin for the treatment of a solid tumor, such as an epithelial cancer, e.g. breast cancer, uterine cancer, ovarian cancer, cervical cancer, vulvar cancer, vaginal cancer, or endometrial cancer. In some embodiments, there are provided pharmaceutical compositions consisting essentially of one or more pharmaceutically acceptable excipients and Apigenin and Luteolin for the treatment of a solid tumor, such as an epithelial cancer, e.g. breast cancer, uterine cancer, ovarian cancer, cervical cancer, vulvar cancer, vaginal cancer, or endometrial cancer. In some embodiments, there are provided pharmaceutical compositions consisting of one or more pharmaceutically acceptable excipients, Apigenin and Luteolin for the treatment of a solid tumor, such as an epithelial cancer, e.g. breast cancer, uterine cancer, ovarian cancer, cervical cancer, vulvar cancer, vaginal cancer, or endometrial cancer.

### DEFINITIONS

As used herein, the term “method” refers to manners, means techniques and procedures for accomplishing a given task including, but not limited to, those manners, means techniques and procedures either known to, or readily developed from known manners, means, techniques and procedures by, practitioners of the chemical, pharmacological, biological, biochemical, medical, and homeopathic arts.

As used herein, “inhibiting the activity” refers to slowing, preferably stopping, the growth and/or proliferation of cancerous cells, both in-place, i.e., growth and proliferation at the initial site of tumor formation, and proliferation by metastasis Inhibiting the activity also encompasses killing cancerous cells.

As used herein, the term “cancer” refers to various types of malignant neoplasms, most of which can invade surrounding tissues, and may metastasize to different sites, as defined by Stedman's Medical Dictionary 25^{th} edition (Hensyl ed. 1990). Examples of cancers which may be treated by the present invention include, but are not limited to, brain, ovarian, colon, prostate, kidney, bladder, breast, lung, oral and skin cancers. In a presently preferred embodiment of this invention the cancer being treated is breast or ovarian cancer.

As used herein, the term “contacting” in the context of contacting a solid tumor cancer cell with an extract of this invention bringing an extract of this invention and a target cancer cell together in such a manner that the extract can affect the activity of the cell either directly or indirectly. As used herein, contacting refers to procedures conducted in vitro, i.e. cancerous cells which are the object of this invention are studied, outside a patient. Cells existing outside the patient can be maintained or grown in cell culture dishes. For cells outside the organism, multiple methods exist, and are well-known to those skilled in the art, to contact extract of this invention, with or without employment of various well-known transmembrane carrier techniques and direct cell microinjection

The term “in vivo” refers to contacting or treatment within a living organism, such as a living human or other mammal, such as a mouse or rat.

As used herein, an “extract” refers to the residue of soluble solids obtained after an herb, or selected part thereof is (1) for example, without limitation, chopped, crushed, pulverized, minced or otherwise treated to expose increased surface area of the plant or plant part to solvent and (2) is placed in intimate contact with a solvent (liquid) to form a mixture, usually, but not necessarily, under conditions of agitation and elevated temperature. Then, after a period of time under the foregoing conditions, the mixture is filtered to remove insoluble solids; and the liquid may be removed by, for example but not limitation, evaporation or freeze drying. The liquid used to obtain an extract may be water or an organic solvent, for example, without limitation, an alcohol such as methyl, ethyl or isopropyl alcohol, a ketone such as acetone or methyl ethyl ketone (MEK), an ester such as ethyl acetate, an organochlorine compound such as methylene chloride, chloroform or carbon tetrachloride, a hydrocarbon such as pentane, hexane or benzene and the like. An extract may also be obtained by using a combination of these solvents with or without water.

As used herein, the terms “treat”, “treating” and “treatment” refer to a method of alleviating or abrogating a solid tumor cancer and/or its attendant symptoms. In particular, the terms simply mean that the life expectancy of an individual affected with a cancer will be increased or that one or more symptoms of the disease will be reduced, thereby enhancing the quality of life (QOL) of the individual treated. Improvement in symptoms may include reduction of tumor size, reduction in rate of tumor growth, reduction in tumor grade, reduction in pain or other symptoms associated with cancer, etc.

As used herein, “administer”, “administering” or “administration” refers to the delivery of a pharmaceutical composition containing of this invention to a patient.

As used herein, the term “mammal” refers to any mammal that is affected by a cancer, whether that cancer is autologous (i.e. arises naturally in the mammal) or is of xenogenous (i.e. xenogenic) origin. The term “mammal” includes humans, as well as murine, canine, feline, equine, bovine, ovine, porcine and other mammalian species.

A “patient” refers to any higher organism that is susceptible to solid tumor cancers. Examples of such higher organisms include, without limitation, mice, rats, rabbits, dogs, cats, horses, cows, pigs, sheep, fish and reptiles. In particular examples, “patient” refers to a human being. In particular embodiments, the human being is a human being suffering from one or more symptoms associated with menopause. In other particular embodiments, the human being is a person suffering from cancer, such as breast cancer, uterine cancer, ovarian cancer, cervical cancer, vulvar cancer, vaginal cancer, endometrial cancer, prostate cancer or other cancer.

As used herein in reference to treatment of cancer, the term “therapeutically effective amount” refers to that amount of a composition according to this invention which has the effect of (1) reducing the size of the tumor; (2) inhibiting (that is, slowing to some extent, preferably stopping) tumor metastasis; (3) inhibiting to some extent (that is slowing to some extent, preferably stopping) tumor growth; and/or; (4) relieving to some extent (or preferably eliminating) one or more symptoms associated with cancer; (5) stabilizing the growth of the tumor; (6) extending the time to disease progression; (7) improving overall survival.

As used herein in reference to treatment of one or more symptoms of menopause, the term “therapeutically effective amount” refers to the amount of a composition according to this invention that has one or more of the following effects: (1) reducing the frequency of such symptom or symptoms; (2) decreasing the severity of such symptom or symptoms; and/or (3) shortening the period of time during which a patient experiences such symptom or symptoms. Symptoms associated with menopause include hot flashes (also known as hot flushes), decreased libido, osteoporosis, vaginal dryness, urinary incontinence, and menopause-associated depression.

As used herein, a “pharmaceutical composition” refers to a mixture of one or more of the compounds or combinations described herein with other chemical components, such as physiologically acceptable carriers and excipients. The purpose of a pharmacological composition is to facilitate administration of composition comprising one or more, two or more, three or more, four or more, five or more, six or all seven of Apigenin, Luteolin, Kaempferol, Narigenin, Quercetin, Quercetrin, and Quercetin-3-O-β-glucopyranoside to patient.

As used herein, the term “pharmaceutically acceptable” means that the referenced agent or excipient is generally regarded as acceptable for use in a pharmaceutical composition.

As used herein, a “physiologically acceptable carrier” refers to a carrier or diluent that does not cause significant irritation to an organism and does not abrogate the biological activity and properties of the administered composition.

As used herein, an “excipient” refers to a pharmaceutically inert substance added to a pharmaceutical composition to further facilitate administration of an extract or extracts of this invention. Examples, without limitation, of excipients include calcium carbonate, calcium phosphate, various sugars and types of starch, cellulose derivatives, gelatin, vegetable oils and polyethylene glycols. The group of excipients and active pharmaceutical ingredients are considered mutually exclusive in the pharmaceutical arts.

As used herein, the terms “comprising”, “comprises”, “comprise” and grammatical variants thereof are inclusive or open-ended and do not exclude additional, unrecited elements or method steps. The terms “include”, “includes”, “contain”, “contains”, “containing” and grammatical variants thereof are likewise inclusive.

As used herein, the phrase “consisting of” excludes any element, step, or ingredient not specified in the following portion of the sentence.

As used herein, the phrase “consisting essentially of” limits the scope of the following part of the sentence to the specified materials or steps and those that do not materially affect the basic and novel characteristic(s) of the claimed invention.

Pharmaceutical Compositions and Modes of Administrations

The compounds and combinations identified herein can be administered to a patient in a mixture comprising at least one suitable excipient. The precise excipient or excipients used will depend upon the route of administration as well as the physical form of the unit dose administered to the patient. In the case of oral elixirs, the excipient may include one or more diluents (e.g. water and/or ethanol), one or more taste masking agents, one or more sweeteners, etc. In the case of oral capsules, the excipient may include one or more gel forming excipients. In the case of intravenous or other parenteral mode of administration, the excipient can include water or saline, which may be adjusted to a suitable pH and/or osmolality for injection.

In treating a patient exhibiting a disorder of interest (e.g. a cancer such as breast cancer or uterine cancer), a therapeutically effective amount of the compound or combination is administered. A therapeutically effective amount refers to that amount of the compound or combination that results in amelioration of symptoms or a prolongation of survival in a patient, and may include destruction of a malignant tumor. Suitable compounds for use herein include Apigenin, Luteolin, Kaempferol, Narigenin, Quercetin, Quercetrin, and Quercetin-3-O-β-glucopyranoside. Suitable combinations of compounds for use herein include two, four, five, six, or all seven of Apigenin, Luteolin, Kaempferol, Narigenin, Quercetin, Quercetrin, and Quercetin-3-O-β-glucopyranoside. In preferred embodiments, the pharmaceutical compositions are at least substantially free of compounds containing an isoprenyl moiety, such as compounds I, II or III, which were described by Yong et al., U.S. Pub. No. 2003/0170292 A1. As used herein, “substantially free” means that the total amount of isoprenyl compounds present in the pharmaceutical compositions is less than 10% of any one of Apigenin, Luteolin, Kaempferol, Narigenin, Quercetin, Quercetrin, and Quercetin-3-O-β-glucopyranoside, and is less than 2% of the total amount of all of Apigenin, Luteolin, Kaempferol, Narigenin, Quercetin, Quercetrin, and/or Quercetin-3-O-β-glucopyranoside that is present in the pharmaceutical composition.

In some embodiments, the pharmaceutical composition comprises Apigenin, and one or more of Kaempferol, Luteolin, Narigenin, Quercetin, Quercetrin, or Quercetin-3-O-β-glucopyranoside.

In some embodiments, the pharmaceutical composition comprises Kaempferol, and one or more of Luteolin, Narigenin, Quercetin, Quercetrin, or Quercetin-3-O-β-glucopyranoside.

In some embodiments, the pharmaceutical composition comprises Luteolin, and one or more of Narigenin, Quercetin, Quercetrin, or Quercetin-3-O-β-glucopyranoside.

In some embodiments, the pharmaceutical composition comprises Narigenin, and one or more of Quercetin, Quercetrin, or Quercetin-3-O-β-glucopyranoside.

In some embodiments, the pharmaceutical composition comprises Quercetin, and one or more of Quercetrin, or Quercetin-3-O-β-glucopyranoside.

In some embodiments, the pharmaceutical composition comprises Quercetrin, and one or more of Quercetin-3-O-β-glucopyranoside.

In some embodiments, the pharmaceutical composition consists essentially of an optional pharmaceutically acceptable excipient, Apigenin, and one or more of Kaempferol, Luteolin, Narigenin, Quercetin, Quercetrin, or Quercetin-3-O-β-glucopyranoside.

In some embodiments, the pharmaceutical composition consists essentially of an optional pharmaceutically acceptable excipient, Kaempferol, and one or more of Luteolin, Narigenin, Quercetin, Quercetrin, or Quercetin-3-O-β-glucopyranoside.

In some embodiments, the pharmaceutical composition consists essentially of an optional pharmaceutically acceptable excipient, Luteolin, and one or more of Narigenin, Quercetin, Quercetrin, or Quercetin-3-O-β-glucopyranoside.

In some embodiments, the pharmaceutical composition consists essentially of an optional pharmaceutically acceptable excipient, Narigenin, and one or more of Quercetin, Quercetrin, or Quercetin-3-O-β-glucopyranoside.

In some embodiments, the pharmaceutical composition consists essentially of an optional pharmaceutically acceptable excipient, Quercetin, and one or more of Quercetrin, or Quercetin-3-O-β-glucopyranoside.

In some embodiments, the pharmaceutical composition consists essentially of an optional pharmaceutically acceptable excipient, Quercetrin, and one or more of Quercetin-3-O-β-glucopyranoside.

In some embodiments, the pharmaceutical composition consists of an optional pharmaceutically acceptable excipient, Apigenin, and one or more of Kaempferol, Luteolin, Narigenin, Quercetin, Quercetrin, or Quercetin-3-O-β-glucopyranoside.

In some embodiments, the pharmaceutical composition consists of an optional pharmaceutically acceptable excipient, Kaempferol, and one or more of Luteolin, Narigenin, Quercetin, Quercetrin, or Quercetin-3-O-β-glucopyranoside.

In some embodiments, the pharmaceutical composition consists of an optional pharmaceutically acceptable excipient, Luteolin, and one or more of Narigenin, Quercetin, Quercetrin, or Quercetin-3-O-β-glucopyranoside.

In some embodiments, the pharmaceutical composition consists of an optional pharmaceutically acceptable excipient, Narigenin, and one or more of Quercetin, Quercetrin, or Quercetin-3-O-β-glucopyranoside.

In some embodiments, the pharmaceutical composition consists of an optional pharmaceutically acceptable excipient, Quercetin, and one or more of Quercetrin, or Quercetin-3-O-β-glucopyranoside. In some embodiments, the pharmaceutical composition consists of an optional pharmaceutically acceptable excipient, Quercetrin, and one or more of Quercetin-3-O-β-glucopyranoside.

In some embodiments, the pharmaceutical composition comprises Apigenin, Kaempferol, and one or more of Luteolin, Narigenin, Quercetin, Quercetrin, or Quercetin-3-O-β-glucopyranoside.

In some embodiments, the pharmaceutical composition comprises Apigenin, Luteolin, and one or more of Narigenin, Quercetin, Quercetrin, or Quercetin-3-O-β-glucopyranoside.

In some embodiments, the pharmaceutical composition Apigenin, Narigenin, and one or more of Quercetin, Quercetrin, or Quercetin-3-O-β-glucopyranoside.

In some embodiments, the pharmaceutical composition comprises Apigenin, Quercetin, and one or more of Quercetrin, or Quercetin-3-O-β-glucopyranoside.

In some embodiments, the pharmaceutical composition comprises In some embodiments, the pharmaceutical composition comprises Apigenin, Quercetrin, and Quercetin-3-O-β-glucopyranoside.

In some embodiments, the pharmaceutical composition comprises Kaempferol, Luteolin, and one or more of Narigenin, Quercetin, Quercetrin, or Quercetin-3-O-β-glucopyranoside.

In some embodiments, the pharmaceutical composition comprises Kaempferol, Narigenin, and one or more of Quercetin, Quercetrin, or Quercetin-3-O-β-glucopyranoside.

In some embodiments, the pharmaceutical composition comprises Kaempferol, Quercetin, and one or more of Quercetrin, or Quercetin-3-O-β-glucopyranoside.

In some embodiments, the pharmaceutical composition comprises Kaempferol, Quercetrin, and Quercetin-3-O-β-glucopyranoside.

In some embodiments, the pharmaceutical composition comprises Luteolin, Narigenin, and one or more of Quercetin, Quercetrin, or Quercetin-3-O-β-glucopyranoside.

In some embodiments, the pharmaceutical composition comprises Luteolin, Quercetin, and one or more of Quercetrin, or Quercetin-3-O-β-glucopyranoside.

In some embodiments, the pharmaceutical composition comprises Luteolin, Quercetrin, and Quercetin-3-O-β-glucopyranoside.

In some embodiments, the pharmaceutical composition comprises Narigenin, Quercetin and one or more of Quercetrin, or Quercetin-3-O-β-glucopyranoside.

In some embodiments, the pharmaceutical composition comprises Narigenin, Quercetrin, and Quercetin-3-O-β-glucopyranoside.

In some embodiments, the pharmaceutical composition comprises Quercetin, Quercetrin, and Quercetin-3-O-β-glucopyranoside.

In some embodiments, the pharmaceutical composition consists essentially of an optional pharmaceutically acceptable excipient, Apigenin, Kaempferol, and one or more of Luteolin, Narigenin, Quercetin, Quercetrin, or Quercetin-3-O-β-glucopyranoside.

In some embodiments, the pharmaceutical composition consists essentially of an optional pharmaceutically acceptable excipient, Apigenin, Luteolin, and one or more of Narigenin, Quercetin, Quercetrin, or Quercetin-3-O-β-glucopyranoside.

In some embodiments, the pharmaceutical composition Apigenin, Narigenin, and one or more of Quercetin, Quercetrin, or Quercetin-3-O-β-glucopyranoside.

In some embodiments, the pharmaceutical composition consists essentially of an optional pharmaceutically acceptable excipient, Apigenin, Quercetin, and one or more of Quercetrin, or Quercetin-3-O-β-glucopyranoside.

In some embodiments, the pharmaceutical composition consists essentially of an optional pharmaceutically acceptable excipient, Apigenin, Quercetrin, and Quercetin-3-O-β-glucopyranoside.

In some embodiments, the pharmaceutical composition consists essentially of an optional pharmaceutically acceptable excipient, Kaempferol, Luteolin, and one or more of Narigenin, Quercetin, Quercetrin, or Quercetin-3-O-β-glucopyranoside.

In some embodiments, the pharmaceutical composition consists essentially of an optional pharmaceutically acceptable excipient, Kaempferol, Narigenin, and one or more of Quercetin, Quercetrin, or Quercetin-3-O-β-glucopyranoside.

In some embodiments, the pharmaceutical composition consists essentially of an optional pharmaceutically acceptable excipient, Kaempferol, Quercetin, and one or more of Quercetrin, or Quercetin-3-O-β-glucopyranoside.

In some embodiments, the pharmaceutical composition consists essentially of an optional pharmaceutically acceptable excipient, Kaempferol, Quercetrin, and Quercetin-3-O-β-glucopyranoside.

In some embodiments, the pharmaceutical composition consists essentially of an optional pharmaceutically acceptable excipient, Luteolin, Narigenin, and one or more of Quercetin, Quercetrin, or Quercetin-3-O-β-glucopyranoside.

In some embodiments, the pharmaceutical composition consists essentially of an optional pharmaceutically acceptable excipient, Luteolin, Quercetin, and one or more of Quercetrin, or Quercetin-3-O-β-glucopyranoside.

In some embodiments, the pharmaceutical composition consists essentially of an optional pharmaceutically acceptable excipient, Luteolin, Quercetrin, and Quercetin-3-O-β-glucopyranoside. In some embodiments, the pharmaceutical composition consists essentially of an optional pharmaceutically acceptable excipient, Narigenin, Quercetin and one or more of Quercetrin, or Quercetin-3-O-β-glucopyranoside.

In some embodiments, the pharmaceutical composition consists essentially of an optional pharmaceutically acceptable excipient, Narigenin, Quercetrin, and Quercetin-3-O-β-glucopyranoside. In some embodiments, the pharmaceutical composition consists essentially of an optional pharmaceutically acceptable excipient, Quercetin, Quercetrin, and Quercetin-3-O-β-glucopyranoside.

In some embodiments, the pharmaceutical composition consists of an optional pharmaceutically acceptable excipient, Apigenin, Kaempferol, and one or more of Luteolin, Narigenin, Quercetin, Quercetrin, or Quercetin-3-O-β-glucopyranoside.

In some embodiments, the pharmaceutical composition consists of an optional pharmaceutically acceptable excipient, Apigenin, Luteolin, and one or more of Narigenin, Quercetin, Quercetrin, or Quercetin-3-O-β-glucopyranoside.

In some embodiments, the pharmaceutical composition Apigenin, Narigenin, and one or more of Quercetin, Quercetrin, or Quercetin-3-O-β-glucopyranoside.

In some embodiments, the pharmaceutical composition consists of an optional pharmaceutically acceptable excipient, Apigenin, Quercetin, and one or more of Quercetrin, or Quercetin-3-O-β-glucopyranoside.

In some embodiments, the pharmaceutical composition consists of an optional pharmaceutically acceptable excipient, Apigenin, Quercetrin, and Quercetin-3-O-β-glucopyranoside.

In some embodiments, the pharmaceutical composition consists of an optional pharmaceutically acceptable excipient, Kaempferol, Luteolin, and one or more of Narigenin, Quercetin, Quercetrin, or Quercetin-3-O-β-glucopyranoside.

In some embodiments, the pharmaceutical composition consists of an optional pharmaceutically acceptable excipient, Kaempferol, Narigenin, and one or more of Quercetin, Quercetrin, or Quercetin-3-O-β-glucopyranoside.

In some embodiments, the pharmaceutical composition consists of an optional pharmaceutically acceptable excipient, Kaempferol, Quercetin, and one or more of Quercetrin, or Quercetin-3-O-β-glucopyranoside.

In some embodiments, the pharmaceutical composition consists of an optional pharmaceutically acceptable excipient, Kaempferol, Quercetrin, and Quercetin-3-O-β-glucopyranoside.

In some embodiments, the pharmaceutical composition consists of an optional pharmaceutically acceptable excipient, Luteolin, Narigenin, and one or more of Quercetin, Quercetrin, or Quercetin-3-O-β-glucopyranoside.

In some embodiments, the pharmaceutical composition consists of an optional pharmaceutically acceptable excipient, Luteolin, Quercetin, and one or more of Quercetrin, or Quercetin-3-O-β-glucopyranoside.

In some embodiments, the pharmaceutical composition consists of an optional pharmaceutically acceptable excipient, Luteolin, Quercetrin, and Quercetin-3-O-β-glucopyranoside.

In some embodiments, the pharmaceutical composition consists of an optional pharmaceutically acceptable excipient, Narigenin, Quercetin and one or more of Quercetrin, or Quercetin-3-O-β-glucopyranoside.

In some embodiments, the pharmaceutical composition consists of an optional pharmaceutically acceptable excipient, Narigenin, Quercetrin, and Quercetin-3-O-β-glucopyranoside.

In some embodiments, the pharmaceutical composition consists of an optional pharmaceutically acceptable excipient, Quercetin, Quercetrin, and Quercetin-3-O-β-glucopyranoside.

In some embodiments, the pharmaceutical composition comprises Apigenin, Kaempferol, Luteolin and one or more of Narigenin, Quercetin, Quercetrin, or Quercetin-3-O-β-glucopyranoside. In some embodiments, the pharmaceutical composition comprises Apigenin, Kaempferol, Narigenin, and one or more of Quercetin, Quercetrin, or Quercetin-3-O-β-glucopyranoside. In some embodiments, the pharmaceutical composition comprises Apigenin, Kaempferol, Quercetin, and one or more of Quercetrin, or Quercetin-3-O-β-glucopyranoside.

In some embodiments, the pharmaceutical composition comprises Apigenin, Kaempferol, Quercetrin, and Quercetin-3-O-β-glucopyranoside.

In some embodiments, the pharmaceutical composition comprises Apigenin, Luteolin, Narigenin, and one or more of Quercetin, Quercetrin, or Quercetin-3-O-β-glucopyranoside.

In some embodiments, the pharmaceutical composition comprises Apigenin, Luteolin, Quercetin, and one or more of Quercetrin, or Quercetin-3-O-β-glucopyranoside.

In some embodiments, the pharmaceutical composition comprises Apigenin, Luteolin, Quercetrin, and Quercetin-3-O-β-glucopyranoside.

In some embodiments, the pharmaceutical composition comprises Apigenin, Narigenin, Quercetin, and one or more of Quercetrin, or Quercetin-3-O-β-glucopyranoside.

In some embodiments, the pharmaceutical composition comprises Apigenin, Narigenin, Quercetrin, and Quercetin-3-O-β-glucopyranoside.

In some embodiments, the pharmaceutical composition comprises Apigenin, Quercetin, Quercetrin, and Quercetin-3-O-β-glucopyranoside.

In some embodiments, the pharmaceutical composition comprises Kaempferol, Luteolin, Narigenin, and one or more of Quercetin, Quercetrin, or Quercetin-3-O-β-glucopyranoside.

In some embodiments, the pharmaceutical composition comprises Kaempferol, Luteolin, Quercetin, and one or more of Quercetrin, or Quercetin-3-O-β-glucopyranoside.

In some embodiments, the pharmaceutical composition comprises Kaempferol, Luteolin, Quercetrin and Quercetin-3-O-β-glucopyranoside.

In some embodiments, the pharmaceutical composition comprises Kaempferol, Narigenin, Quercetin, and one or more of Quercetrin, or Quercetin-3-O-β-glucopyranoside.

In some embodiments, the pharmaceutical composition comprises Kaempferol, Narigenin, Quercetrin, and Quercetin-3-O-β-glucopyranoside.

In some embodiments, the pharmaceutical composition comprises Kaempferol, Quercetin, Quercetrin, and Quercetin-3-O-β-glucopyranoside.

In some embodiments, the pharmaceutical composition comprises Luteolin, Narigenin, Quercetin, and one or more of Quercetrin, or Quercetin-3-O-β-glucopyranoside.

In some embodiments, the pharmaceutical composition comprises Luteolin, Narigenin, Quercetrin, and Quercetin-3-O-β-glucopyranoside.

In some embodiments, the pharmaceutical composition comprises Luteolin, Quercetin, Quercetrin, and Quercetin-3-O-β-glucopyranoside.

In some embodiments, the pharmaceutical composition comprises Narigenin, Quercetin, Quercetrin, and Quercetin-3-O-β-glucopyranoside.

In some embodiments, the pharmaceutical composition consists essentially of an optional pharmaceutically acceptable excipient, Apigenin, Kaempferol, Luteolin and one or more of Narigenin, Quercetin, Quercetrin, or Quercetin-3-O-β-glucopyranoside.

In some embodiments, the pharmaceutical composition consists essentially of an optional pharmaceutically acceptable excipient, Apigenin, Kaempferol, Narigenin, and one or more of Quercetin, Quercetrin, or Quercetin-3-O-β-glucopyranoside.

In some embodiments, the pharmaceutical composition consists essentially of an optional pharmaceutically acceptable excipient, Apigenin, Kaempferol, Quercetin, and one or more of Quercetrin, or Quercetin-3-O-β-glucopyranoside.

In some embodiments, the pharmaceutical composition consists essentially of an optional pharmaceutically acceptable excipient, Apigenin, Kaempferol, Quercetrin, and Quercetin-3-O-β-glucopyranoside.

In some embodiments, the pharmaceutical composition consists essentially of an optional pharmaceutically acceptable excipient, Apigenin, Luteolin, Narigenin, and one or more of Quercetin, Quercetrin, or Quercetin-3-O-β-glucopyranoside.

In some embodiments, the pharmaceutical composition consists essentially of an optional pharmaceutically acceptable excipient, Apigenin, Luteolin, Quercetin, and one or more of Quercetrin, or Quercetin-3-O-β-glucopyranoside.

In some embodiments, the pharmaceutical composition consists essentially of an optional pharmaceutically acceptable excipient, Apigenin, Luteolin, Quercetrin, and Quercetin-3-O-β-glucopyranoside.

In some embodiments, the pharmaceutical composition consists essentially of an optional pharmaceutically acceptable excipient, Apigenin, Narigenin, Quercetin, and one or more of Quercetrin, or Quercetin-3-O-β-glucopyranoside.

In some embodiments, the pharmaceutical composition consists essentially of an optional pharmaceutically acceptable excipient, Apigenin, Narigenin, Quercetrin, and Quercetin-3-O-β-glucopyranoside.

In some embodiments, the pharmaceutical composition consists essentially of an optional pharmaceutically acceptable excipient, Apigenin, Quercetin, Quercetrin, and Quercetin-3-O-β-glucopyranoside.

In some embodiments, the pharmaceutical composition consists essentially of an optional pharmaceutically acceptable excipient, Kaempferol, Luteolin, Narigenin, and one or more of Quercetin, Quercetrin, or Quercetin-3-O-β-glucopyranoside.

In some embodiments, the pharmaceutical composition consists essentially of an optional pharmaceutically acceptable excipient, Kaempferol, Luteolin, Quercetin, and one or more of Quercetrin, or Quercetin-3-O-β-glucopyranoside.

In some embodiments, the pharmaceutical composition consists essentially of an optional pharmaceutically acceptable excipient, Kaempferol, Luteolin, Quercetrin and Quercetin-3-O-β-glucopyranoside.

In some embodiments, the pharmaceutical composition consists essentially of an optional pharmaceutically acceptable excipient, Kaempferol, Narigenin, Quercetin, and one or more of Quercetrin, or Quercetin-3-O-β-glucopyranoside.

In some embodiments, the pharmaceutical composition consists essentially of an optional pharmaceutically acceptable excipient, Kaempferol, Narigenin, Quercetrin, and Quercetin-3-O-β-glucopyranoside.

In some embodiments, the pharmaceutical composition consists essentially of an optional pharmaceutically acceptable excipient, Kaempferol, Quercetin, Quercetrin, and Quercetin-3-O-β-glucopyranoside.

In some embodiments, the pharmaceutical composition consists essentially of an optional pharmaceutically acceptable excipient, Luteolin, Narigenin, Quercetin, and one or more of Quercetrin, or Quercetin-3-O-β-glucopyranoside.

In some embodiments, the pharmaceutical composition consists essentially of an optional pharmaceutically acceptable excipient, Luteolin, Narigenin, Quercetrin, and Quercetin-3-O-β-glucopyranoside.

In some embodiments, the pharmaceutical composition consists essentially of an optional pharmaceutically acceptable excipient, Luteolin, Quercetin, Quercetrin, and Quercetin-3-O-β-glucopyranoside.

In some embodiments, the pharmaceutical composition consists essentially of an optional pharmaceutically acceptable excipient, Narigenin, Quercetin, Quercetrin, and Quercetin-3-O-β-glucopyranoside.

In some embodiments, the pharmaceutical composition consists of an optional pharmaceutically acceptable excipient, Apigenin, Kaempferol, Luteolin and one or more of Narigenin, Quercetin, Quercetrin, or Quercetin-3-O-β-glucopyranoside.

In some embodiments, the pharmaceutical composition consists of an optional pharmaceutically acceptable excipient, Apigenin, Kaempferol, Narigenin, and one or more of Quercetin, Quercetrin, or Quercetin-3-O-β-glucopyranoside.

In some embodiments, the pharmaceutical composition consists of an optional pharmaceutically acceptable excipient, Apigenin, Kaempferol, Quercetin, and one or more of Quercetrin, or Quercetin-3-O-β-glucopyranoside.

In some embodiments, the pharmaceutical composition consists of an optional pharmaceutically acceptable excipient, Apigenin, Kaempferol, Quercetrin, and Quercetin-3-O-β-glucopyranoside.

In some embodiments, the pharmaceutical composition consists of an optional pharmaceutically acceptable excipient, Apigenin, Luteolin, Narigenin, and one or more of Quercetin, Quercetrin, or Quercetin-3-O-β-glucopyranoside.

In some embodiments, the pharmaceutical composition consists of an optional pharmaceutically acceptable excipient, Apigenin, Luteolin, Quercetin, and one or more of Quercetrin, or Quercetin-3-O-β-glucopyranoside.

In some embodiments, the pharmaceutical composition consists of an optional pharmaceutically acceptable excipient, Apigenin, Luteolin, Quercetrin, and Quercetin-3-O-β-glucopyranoside.

In some embodiments, the pharmaceutical composition consists of an optional pharmaceutically acceptable excipient, Apigenin, Narigenin, Quercetin, and one or more of Quercetrin, or Quercetin-3-O-β-glucopyranoside.

In some embodiments, the pharmaceutical composition consists of an optional pharmaceutically acceptable excipient, Apigenin, Narigenin, Quercetrin, and Quercetin-3-O-β-glucopyranoside.

In some embodiments, the pharmaceutical composition consists of an optional pharmaceutically acceptable excipient, Apigenin, Quercetin, Quercetrin, and Quercetin-3-O-β-glucopyranoside.

In some embodiments, the pharmaceutical composition consists of an optional pharmaceutically acceptable excipient, Kaempferol, Luteolin, Narigenin, and one or more of Quercetin, Quercetrin, or Quercetin-3-O-β-glucopyranoside.

In some embodiments, the pharmaceutical composition consists of an optional pharmaceutically acceptable excipient, Kaempferol, Luteolin, Quercetin, and one or more of Quercetrin, or Quercetin-3-O-β-glucopyranoside.

In some embodiments, the pharmaceutical composition consists of an optional pharmaceutically acceptable excipient, Kaempferol, Luteolin, Quercetrin and Quercetin-3-O-β-glucopyranoside.

In some embodiments, the pharmaceutical composition consists of an optional pharmaceutically acceptable excipient, Kaempferol, Narigenin, Quercetin, and one or more of Quercetrin, or Quercetin-3-O-β-glucopyranoside.

In some embodiments, the pharmaceutical composition consists of an optional pharmaceutically acceptable excipient, Kaempferol, Narigenin, Quercetrin, and Quercetin-3-O-β-glucopyranoside.

In some embodiments, the pharmaceutical composition consists of an optional pharmaceutically acceptable excipient, Kaempferol, Quercetin, Quercetrin, and Quercetin-3-O-β-glucopyranoside.

In some embodiments, the pharmaceutical composition consists of an optional pharmaceutically acceptable excipient, Luteolin, Narigenin, Quercetin, and one or more of Quercetrin, or Quercetin-3-O-β-glucopyranoside.

In some embodiments, the pharmaceutical composition consists of an optional pharmaceutically acceptable excipient, Luteolin, Narigenin, Quercetrin, and Quercetin-3-O-β-glucopyranoside.

In some embodiments, the pharmaceutical composition consists of an optional pharmaceutically acceptable excipient, Luteolin, Quercetin, Quercetrin, and Quercetin-3-O-β-glucopyranoside.

In some embodiments, the pharmaceutical composition consists of an optional pharmaceutically acceptable excipient, Narigenin, Quercetin, Quercetrin, and Quercetin-3-O-β-glucopyranoside.

In some embodiments, the pharmaceutical composition comprises Apigenin, Kaempferol, Luteolin, Narigenin, and Quercetin, Quercetrin, or Quercetin-3-O-β-glucopyranoside.

In some embodiments, the pharmaceutical composition comprises Apigenin, Kaempferol, Luteolin, Quercetin, and Quercetrin, or Quercetin-3-O-β-glucopyranoside.

In some embodiments, the pharmaceutical composition comprises Apigenin, Kaempferol, Luteolin, Quercetrin, and Quercetin-3-O-β-glucopyranoside.

In some embodiments, the pharmaceutical composition comprises Apigenin, Kaempferol, Narigenin, Quercetin, and Quercetrin, or Quercetin-3-O-β-glucopyranoside.

In some embodiments, the pharmaceutical composition comprises Apigenin, Kaempferol, Narigenin, Quercetrin, and Quercetin-3-O-β-glucopyranoside.

In some embodiments, the pharmaceutical composition comprises Apigenin, Kaempferol, Quercetin, Quercetrin, and Quercetin-3-O-β-glucopyranoside.

In some embodiments, the pharmaceutical composition comprises Apigenin, Luteolin, Narigenin, Quercetin, and Quercetrin, or Quercetin-3-O-β-glucopyranoside.

In some embodiments, the pharmaceutical composition comprises Apigenin, Luteolin, Narigenin, Quercetrin, and Quercetin-3-O-β-glucopyranoside.

In some embodiments, the pharmaceutical composition comprises Apigenin, Luteolin, Quercetin, Quercetrin, and Quercetin-3-O-β-glucopyranoside.

In some embodiments, the pharmaceutical composition comprises Apigenin, Narigenin, Quercetin, Quercetrin, and Quercetin-3-O-β-glucopyranoside.

In some embodiments, the pharmaceutical composition comprises Kaempferol, Luteolin, Narigenin, Quercetin, and Quercetrin, or Quercetin-3-O-β-glucopyranoside.

In some embodiments, the pharmaceutical composition comprises Kaempferol, Luteolin, Quercetin, Quercetrin, and Quercetin-3-O-β-glucopyranoside.

In some embodiments, the pharmaceutical composition comprises Kaempferol, Luteolin, Narigenin, Quercetrin, and Quercetin-3-O-β-glucopyranoside.

In some embodiments, the pharmaceutical composition comprises Kaempferol, Narigenin, Quercetin, Quercetrin, and Quercetin-3-O-β-glucopyranoside.

In some embodiments, the pharmaceutical composition comprises Luteolin, Narigenin, Quercetin, Quercetrin and Quercetin-3-O-β-glucopyranoside.

In some embodiments, the pharmaceutical composition consists essentially of an optional pharmaceutically acceptable excipient, Apigenin, Kaempferol, Luteolin, Narigenin, and Quercetin, Quercetrin, or Quercetin-3-O-β-glucopyranoside.

In some embodiments, the pharmaceutical composition consists essentially of an optional pharmaceutically acceptable excipient, Apigenin, Kaempferol, Luteolin, Quercetin, and Quercetrin, or Quercetin-3-O-β-glucopyranoside.

In some embodiments, the pharmaceutical composition consists essentially of an optional pharmaceutically acceptable excipient, Apigenin, Kaempferol, Luteolin, Quercetrin, and Quercetin-3-O-β-glucopyranoside.

In some embodiments, the pharmaceutical composition consists essentially of an optional pharmaceutically acceptable excipient, Apigenin, Kaempferol, Narigenin, Quercetin, and Quercetrin, or Quercetin-3-O-β-glucopyranoside.

In some embodiments, the pharmaceutical composition consists essentially of an optional pharmaceutically acceptable excipient, Apigenin, Kaempferol, Narigenin, Quercetrin, and Quercetin-3-O-β-glucopyranoside.

In some embodiments, the pharmaceutical composition consists essentially of an optional pharmaceutically acceptable excipient, Apigenin, Kaempferol, Quercetin, Quercetrin, and Quercetin-3-O-β-glucopyranoside.

In some embodiments, the pharmaceutical composition consists essentially of an optional pharmaceutically acceptable excipient, Apigenin, Luteolin, Narigenin, Quercetin, and Quercetrin, or Quercetin-3-O-β-glucopyranoside.

In some embodiments, the pharmaceutical composition consists essentially of an optional pharmaceutically acceptable excipient, Apigenin, Luteolin, Narigenin, Quercetrin, and Quercetin-3-O-β-glucopyranoside.

In some embodiments, the pharmaceutical composition consists essentially of an optional pharmaceutically acceptable excipient, Apigenin, Luteolin, Quercetin, Quercetrin, and Quercetin-3-O-β-glucopyranoside.

In some embodiments, the pharmaceutical composition consists essentially of an optional pharmaceutically acceptable excipient, Apigenin, Narigenin, Quercetin, Quercetrin, and Quercetin-3-O-β-glucopyranoside.

In some embodiments, the pharmaceutical composition consists essentially of an optional pharmaceutically acceptable excipient, Kaempferol, Luteolin, Narigenin, Quercetin, and Quercetrin, or Quercetin-3-O-β-glucopyranoside.

In some embodiments, the pharmaceutical composition consists essentially of an optional pharmaceutically acceptable excipient, Kaempferol, Luteolin, Quercetin, Quercetrin, and Quercetin-3-O-β-glucopyranoside.

In some embodiments, the pharmaceutical composition consists essentially of an optional pharmaceutically acceptable excipient, Kaempferol, Luteolin, Narigenin, Quercetrin, and Quercetin-3-O-β-glucopyranoside.

In some embodiments, the pharmaceutical composition consists essentially of an optional pharmaceutically acceptable excipient, Kaempferol, Narigenin, Quercetin, Quercetrin, and Quercetin-3-O-β-glucopyranoside.

In some embodiments, the pharmaceutical composition consists essentially of an optional pharmaceutically acceptable excipient, Luteolin, Narigenin, Quercetin, Quercetrin and Quercetin-3-O-β-glucopyranoside.

In some embodiments, the pharmaceutical composition consists of an optional pharmaceutically acceptable excipient, Apigenin, Kaempferol, Luteolin, Narigenin, and Quercetin, Quercetrin, or Quercetin-3-O-β-glucopyranoside.

In some embodiments, the pharmaceutical composition consists of an optional pharmaceutically acceptable excipient, Apigenin, Kaempferol, Luteolin, Quercetin, and Quercetrin, or Quercetin-3-O-β-glucopyranoside.

In some embodiments, the pharmaceutical composition consists of an optional pharmaceutically acceptable excipient, Apigenin, Kaempferol, Luteolin, Quercetrin, and Quercetin-3-O-β-glucopyranoside.

In some embodiments, the pharmaceutical composition consists of an optional pharmaceutically acceptable excipient, Apigenin, Kaempferol, Narigenin, Quercetin, and Quercetrin, or Quercetin-3-O-β-glucopyranoside.

In some embodiments, the pharmaceutical composition consists of an optional pharmaceutically acceptable excipient, Apigenin, Kaempferol, Narigenin, Quercetrin, and Quercetin-3-O-β-glucopyranoside.

In some embodiments, the pharmaceutical composition consists of an optional pharmaceutically acceptable excipient, Apigenin, Kaempferol, Quercetin, Quercetrin, and Quercetin-3-O-β-glucopyranoside.

In some embodiments, the pharmaceutical composition consists of an optional pharmaceutically acceptable excipient, Apigenin, Luteolin, Narigenin, Quercetin, and Quercetrin, or Quercetin-3-O-β-glucopyranoside.

In some embodiments, the pharmaceutical composition consists of an optional pharmaceutically acceptable excipient, Apigenin, Luteolin, Narigenin, Quercetrin, and Quercetin-3-O-β-glucopyranoside.

In some embodiments, the pharmaceutical composition consists of an optional pharmaceutically acceptable excipient, Apigenin, Luteolin, Quercetin, Quercetrin, and Quercetin-3-O-β-glucopyranoside.

In some embodiments, the pharmaceutical composition consists of an optional pharmaceutically acceptable excipient, Apigenin, Narigenin, Quercetin, Quercetrin, and Quercetin-3-O-β-glucopyranoside.

In some embodiments, the pharmaceutical composition consists of an optional pharmaceutically acceptable excipient, Kaempferol, Luteolin, Narigenin, Quercetin, and Quercetrin, or Quercetin-3-O-β-glucopyranoside.

In some embodiments, the pharmaceutical composition consists of an optional pharmaceutically acceptable excipient, Kaempferol, Luteolin, Quercetin, Quercetrin, and Quercetin-3-O-β-glucopyranoside.

In some embodiments, the pharmaceutical composition consists of an optional pharmaceutically acceptable excipient, Kaempferol, Luteolin, Narigenin, Quercetrin, and Quercetin-3-O-β-glucopyranoside.

In some embodiments, the pharmaceutical composition consists of an optional pharmaceutically acceptable excipient, Kaempferol, Narigenin, Quercetin, Quercetrin, and Quercetin-3-O-β-glucopyranoside.

In some embodiments, the pharmaceutical composition consists of an optional pharmaceutically acceptable excipient, Luteolin, Narigenin, Quercetin, Quercetrin and Quercetin-3-O-β-glucopyranoside.

In some embodiments, the pharmaceutical composition comprises Apigenin, Kaempferol, Luteolin, Narigenin, Quercetin, and Quercetrin, or Quercetin-3-O-β-glucopyranoside.

In some embodiments, the pharmaceutical composition consists of an optional pharmaceutically acceptable excipient, Apigenin, Kaempferol, Luteolin, Narigenin, Quercetrin, and Quercetin-3-O-β-glucopyranoside.

In some embodiments, the pharmaceutical composition comprises Apigenin, Kaempferol, Luteolin, Quercetin, Quercetrin, and Quercetin-3-O-β-glucopyranoside.

In some embodiments, the pharmaceutical composition comprises Apigenin, Kaempferol, Narigenin, Quercetin, Quercetrin, and Quercetin-3-O-β-glucopyranoside.

In some embodiments, the pharmaceutical composition comprises Apigenin, Luteolin, Narigenin, Quercetin, Quercetrin, and Quercetin-3-O-β-glucopyranoside.

In some embodiments, the pharmaceutical composition comprises Kaempferol, Luteolin, Narigenin, Quercetin, Quercetrin, and Quercetin-3-O-β-glucopyranoside.

In some embodiments, the pharmaceutical composition comprises Apigenin, Kaempferol, Luteolin, Narigenin, Quercetin, Quercetrin, and Quercetin-3-O-β-glucopyranoside.

In some embodiments, the pharmaceutical composition consists essentially of an optional pharmaceutically acceptable excipient, Apigenin, Kaempferol, Luteolin, Narigenin, Quercetin, and Quercetrin, or Quercetin-3-O-β-glucopyranoside.

In some embodiments, the pharmaceutical composition consists essentially of an optional pharmaceutically acceptable excipient, Apigenin, Kaempferol, Luteolin, Narigenin, Quercetrin, and Quercetin-3-O-β-glucopyranoside.

In some embodiments, the pharmaceutical composition consists essentially of an optional pharmaceutically acceptable excipient, Apigenin, Kaempferol, Luteolin, Quercetin, Quercetrin, and Quercetin-3-O-β-glucopyranoside.

In some embodiments, the pharmaceutical composition consists essentially of an optional pharmaceutically acceptable excipient, Apigenin, Kaempferol, Narigenin, Quercetin, Quercetrin, and Quercetin-3-O-β-glucopyranoside.

In some embodiments, the pharmaceutical composition consists essentially of an optional pharmaceutically acceptable excipient, Apigenin, Luteolin, Narigenin, Quercetin, Quercetrin, and Quercetin-3-O-β-glucopyranoside.

In some embodiments, the pharmaceutical composition consists essentially of an optional pharmaceutically acceptable excipient, Kaempferol, Luteolin, Narigenin, Quercetin, Quercetrin, and Quercetin-3-O-β-glucopyranoside.

In some embodiments, the pharmaceutical composition consists essentially of an optional pharmaceutically acceptable excipient, Apigenin, Kaempferol, Luteolin, Narigenin, Quercetin, Quercetrin, and Quercetin-3-O-β-glucopyranoside.

In some embodiments, the pharmaceutical composition consists of an optional pharmaceutically acceptable excipient, Apigenin, Kaempferol, Luteolin, Narigenin, Quercetin, and Quercetrin, or Quercetin-3-O-β-glucopyranoside.

In some embodiments, the pharmaceutical composition consists of an optional pharmaceutically acceptable excipient, Apigenin, Kaempferol, Luteolin, Narigenin, Quercetrin, and Quercetin-3-O-β-glucopyranoside.

In some embodiments, the pharmaceutical composition consists of an optional pharmaceutically acceptable excipient, Apigenin, Kaempferol, Luteolin, Quercetin, Quercetrin, and Quercetin-3-O-β-glucopyranoside.

In some embodiments, the pharmaceutical composition consists of an optional pharmaceutically acceptable excipient, Apigenin, Kaempferol, Narigenin, Quercetin, Quercetrin, and Quercetin-3-O-β-glucopyranoside.

In some embodiments, the pharmaceutical composition consists of an optional pharmaceutically acceptable excipient, Apigenin, Luteolin, Narigenin, Quercetin, Quercetrin, and Quercetin-3-O-β-glucopyranoside.

In some embodiments, the pharmaceutical composition consists of an optional pharmaceutically acceptable excipient, Kaempferol, Luteolin, Narigenin, Quercetin, Quercetrin, and Quercetin-3-O-β-glucopyranoside.

In some embodiments, the pharmaceutical composition consists of an optional pharmaceutically acceptable excipient, Apigenin, Kaempferol, Luteolin, Narigenin, Quercetin, Quercetrin, and Quercetin-3-O-β-glucopyranoside.

In some preferred embodiments, the pharmaceutical compositions optionally contain an pharmaceutically acceptable excipient, and also contain each of Apigenin, Kaempferol, Luteolin, Narigenin, Quercetin, Quercetrin, and Quercetin-3-O-β-glucopyranoside. In particularly preferred embodiments, the pharmaceutical composition contains each of Apigenin, Kaempferol, Luteolin, Narigenin, Quercetin, Quercetrin, and Quercetin-3-O-β-glucopyranoside, and is substantially free of compounds containing an isoprenyl moiety, such as compounds I, II or III, which were described by Yong et al., U.S. Pub. No. 2003/0170292 A1.

It will be recognized that Apigenin, Luteolin, Kaempferol, Narigenin, Quercetin, Quercetrin, and Quercetin-3-O-β-glucopyranoside may exist in a variety of physical forms, including free base, salt, crystalline, amorphous, hydrate, and other physical forms. Unless otherwise specified herein, the terms Apigenin, Luteolin, Kaempferol, Narigenin, Quercetin, Quercetrin, and Quercetin-3-O-β-glucopyranoside, are intended to include these additional physical forms. Thus, as a non-limiting, illustrative example, in some embodiments, a pharmaceutical composition that comprises, consists essentially of, or consists of, at least one member of the group consisting of Apigenin, Luteolin, Kaempferol, Narigenin, Quercetin, Quercetrin, and Quercetin-3-O-β-glucopyranoside, contains a pharmaceutically acceptable salt of at least one member of the group consisting of Apigenin, Luteolin, Kaempferol, Narigenin, Quercetin, Quercetrin, and Quercetin-3-O-β-glucopyranoside. In some other illustrative embodiments, a pharmaceutical composition that comprises, consists essentially of, or consists of, at least one member of the group consisting of Apigenin, Luteolin, Kaempferol, Narigenin, Quercetin, Quercetrin, and Quercetin-3-O-β-glucopyranoside contains no salt form of any of group consisting of Apigenin, Luteolin, Kaempferol, Narigenin, Quercetin, Quercetrin, and Quercetin-3-O-β-glucopyranoside. In some embodiments in which the pharmaceutical composition contains one or more of Apigenin free base, Luteolin free base, a pharmaceutically acceptable salt of Apigenin, or a pharmaceutically acceptable salt of Luteolin, the pharmaceutical composition contains at least one member of the group consisting of Kaempferol (free base or pharmaceutically acceptable salt), Narigenin (free base or pharmaceutically acceptable salt), Quercetin (free base or pharmaceutically acceptable salt), Quercetrin (free base or pharmaceutically acceptable salt), and Quercetin-3-O-β-glucopyranoside (free base or pharmaceutically acceptable salt).

When administered without combination with any other substances, the composition comprising a compound or combination described herein may be encased in a suitable capsule, such as a gelatin capsule. When administered in admixture with other excipients, adjuvants, binders, diluents, disintegrants, etc., the compound or combination may be compressed into a capsule or caplet in a conventional manner that is well-known in the art.

Administration of Compounds and Combinations

Toxicity and therapeutic efficacy of the herein-described compounds and combinations, i.e., determining the LD50 (the dose lethal to 50% of the population) and the ED50 (the dose therapeutically effective in 50% of the population) can be determined by standard pharmaceutical procedures in cell cultures or experimental animals. The dose ratio between toxic and therapeutic effects is the therapeutic index and it can be expressed as the ratio LD50/ED50. Compounds and combinations that exhibit large therapeutic indices are preferred. The data obtained from these cell culture assays and animal studies can be used in formulating a range of dosages for use in humans, in particular for internal use, that include ED50 with little or no toxicity. The dosage may vary within this range depending upon the dosage form employed and the route of administration utilized.

For any compound or combination used in the method of invention, the therapeutically effective dose can be estimated initially from cell culture assays. For example, a dose can be formulated in animal models to achieve a circulating plasma concentration range that includes the IC50 as determined in cell culture. Such information can be used to more accurately determine useful doses in humans. Levels in plasma may be measured, for example, by HPLC.

It should be noted that the attending physician would know how and when to terminate, interrupt, or adjust administration due to toxicity, or organ dysfunction. Conversely, the attending physician would also know to adjust treatment to higher levels if the clinical response is not adequate. The severity of the condition may, for example, be evaluated, in part, by standard prognostic evaluation methods. Further, the dose and perhaps dose frequency will also vary according to the age, body weight, and response of the individual patient. A program comparable to that discussed above may be used in veterinary medicine.

If desired, standard western medicine techniques for formulation and administration may be used, such as those found in Remington's Pharmaceutical Sciences, 18^{th} ed., Mack Publishing Co., Easton, Pa. (1990). Suitable routes may include: oral, rectal, transdermal, vaginal, transmucosal, or intestinal administration; parenteral delivery, including intramuscular, subcutaneous, intramedullary injections; as well as intrathecal, direct intraventricular, intravenous, intraperitoneal, intranasal, or intraocular injections, to name a just a few. In particular embodiments, the compounds and combinations of the invention are administered orally.

For injection a compound or composition as described herein may be formulated in aqueous solutions, preferably in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate a compound or composition as described herein in the methods disclosed for the practice of this invention in dosages suitable for systemic administration is within the scope of the invention. With proper choice of carrier and suitable manufacturing practice, a compound or composition as described herein, in particular those formulated as solutions, may be administered parenterally, such as by intravenous injection. Likewise, a compound or composition as described herein can be formulated, using pharmaceutically acceptable carriers well known in the art, into dosages suitable for oral administration. Such carriers enable a compound or composition as described herein to be formulated as tablets, pills, capsules, liquids, gels, syrups, slurries, suspensions and the like, for oral ingestion by a patient to be treated.

Pharmaceutical compositions suitable for use in the present invention are compositions wherein a compound or composition as described herein is contained in an effective amount to achieve its intended purpose. Determination of the effective amount is well within the capability of those skilled in the art, especially in light of the detailed disclosure provided herein. A pharmaceutical composition may contain suitable pharmaceutically acceptable carriers including excipients and auxiliaries that facilitate processing of a compound or composition as described herein into preparations that can be used pharmaceutically. The preparations formulated for oral administration may be in the form of tablets, dragees, capsules, or solutions. The pharmaceutical compositions may be manufactured in a known manner e.g., by means of conventional mixing, dissolving, granulating, dragee-making, levitating, emulsifying, encapsulating, entrapping or lyophilizing processes.

Pharmaceutically formulations for parenteral administration include aqueous solutions of a compound or composition as described herein in water-soluble form. Additionally, suspensions of a compound or composition as described herein may be prepared as appropriate oily injection suspensions may contain substances that increase the viscosity of the suspension, such as sodium carboxymethyl cellulose, sorbitol, or dextran. Optionally, the suspension may also contain suitable stabilizers or agents that increase the solubility of an extract to allow for the preparation of highly concentrated solutions.

Pharmaceutical preparations for oral use can be obtained by combining a compound or composition as described herein with solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum Arabic, talc, polyvinyl pyrrolidone, carpool gel, polyethylene glycol, and/or titanium dioxide, lacquer solutions, and suitable organic solvents or solvent mixtures. Dyestuffs or pigments may be added to the tablets or dragee coatings for identification or to characterize different combinations of extracts and/or doses.

Pharmaceutical preparations that can be used orally include push-fit capsules made of gelatin, as well as soft, sealed capsules made of gelatin and a plasticizer, such as glycerol or sorbitol. The push-fit capsules contain a compound or composition as described herein in admixture with fillers such as lactose, binders such as starches, and/or lubricants such as talc or magnesium separate and, optionally, stabilizers. In soft capsules, a compound or composition as described herein may be dissolved or suspended in suitable liquids, such as fatty oils, liquid paraffin, or liquid polyethylene glycols.

The dosage of a compound or composition as described herein varies depending upon the tumor type, the stage of disease, the species of patient and the individual patient. In general, the amount of a compound or composition as described herein administered to a human patient is about 0.1 mg to about 100 g (in particular about 1 mg to about 50 g, or about 10 mg to about 30 g) of a one or a combination of two or more of Apigenin, Kaempferol, Luteolin, Narigenin, Quercetin, Quercetrin, and Quercetin-3-O-β-glucopyranoside. Some embodiments herein provide a dose of about 0.1 mg to about 10000 mg of one, two, four, five or more, six, or all seven of the members of the group consisting of Apigenin, Kaempferol, Luteolin, Narigenin, Quercetin, Quercetrin, and Quercetin-3-O-β-glucopyranoside. (E.g. in a combination comprising all seven of the actives, the composition contains 0.1 mg to 10,000 mg of Apigenin, 0.1 mg to 10,000 mg of Kaempferol, 0.1 mg to 10,000 mg of Luteolin, 0.1 to 10,000 mg of Narigenin, 0.1 to 10,000 mg of Quercetin, 0.1 mg to 10,000 mg of Quercetrin, and 0.1 mg to 10,000 mg of Quercetin-3-O-(3-glucopyranoside.) In some embodiments, the effective dose is equivalent to about 1 mg to about 5000 mg of, two, four, five or more, six, or all seven of the of Apigenin, Kaempferol, Luteolin, Narigenin, Quercetin, Quercetrin, and Quercetin-3-O-β-glucopyranoside. In some embodiments, the effective dose is equivalent to about 10 mg to about 5000 mg of, two, four, five or more, six, or all seven of the of Apigenin, Kaempferol, Luteolin, Narigenin, Quercetin, Quercetrin, and Quercetin-3-O-β-glucopyranoside. In some embodiments, the effective dose is equivalent to about 100 mg to about 5000 mg of, two, four, five or more, six, or all seven of the of Apigenin, Kaempferol, Luteolin, Narigenin, Quercetin, Quercetrin, and Quercetin-3-O-β-glucopyranoside. In some embodiments, the effective dose is equivalent to about 1000 mg to about 5000 mg of, two, four, five or more, six, or all seven of the of Apigenin, Kaempferol, Luteolin, Narigenin, Quercetin, Quercetrin, and Quercetin-3-O-β-glucopyranoside. In some embodiments, the dose is about 5 to about 250 mg of one, two, four, five or more, six, or all seven of Apigenin, Kaempferol, Luteolin, Narigenin, Quercetin, Quercetrin, and Quercetin-3-O-β-glucopyranoside.

Some embodiments of the invention include compositions that contain one, two, three, four, five, six or all seven of Apigenin, Luteolin, Kaempferol, Narigenin, Quercetin, Quercetrin, and Quercetin-3-O-β-glucopyranoside, and contain no appreciable amount of a compound containing an isoprenyl moiety, such as compounds I, II or III, which were described by Yong et al., U.S. Pub. No. 2003/0170292 A1. In this context, “no appreciable amount” means that the total amount of isoprenyl compound or compounds present in the composition is less than 1% of the total amount of Apigenin, Luteolin, Kaempferol, Narigenin, Quercetin, Quercetrin, and/or Quercetin-3-O-β-glucopyranoside present in the composition.

In some embodiments, the compositions contain one, two, three, four, five, six or all seven of Apigenin, Luteolin, Kaempferol, Narigenin, Quercetin, Quercetrin, and Quercetin-3-O-β-glucopyranoside, and contain substantially no compound containing an isoprenyl moiety, such as compounds I, II or III, which were described by Yong et al., U.S. Pub. No. 2003/0170292 A1. In this context, “substantially no” means that the total amount of isoprenyl compounds in a composition is less than 0.1% of the total amount of Apigenin, Luteolin, Kaempferol, Narigenin, Quercetin, Quercetrin, and/or Quercetin-3-O-β-glucopyranoside present in the composition.

In some embodiments, the compositions contain one, two, three, four, five, six or all of Apigenin, Luteolin, Kaempferol, Narigenin, Quercetin, Quercetrin, and Quercetin-3-O-β-glucopyranoside, and contain no compound containing an isoprenyl moiety, such as compounds I, II or III, which were described by Yong et al., U.S. Pub. No. 2003/0170292 A1. In this context, “no” means that the total amount of isoprenyl compounds in the composition is less than 0.05% of the total amount of Apigenin, Luteolin, Kaempferol, Narigenin, Quercetin, Quercetrin, and/or Quercetin-3-O-β-glucopyranoside present in the composition.

In some embodiments, the compositions contain one, two, three, four, five, six or all of Apigenin, Luteolin, Kaempferol, Narigenin, Quercetin, Quercetrin, and Quercetin-3-O-β-glucopyranoside, and contain an undetectable amount of compound containing an isoprenyl moiety, such as compounds I, II or III, which were described by Yong et al., U.S. Pub. No. 2003/0170292 A1.

### EXAMPLES

### Preparative Example 1

### Isolation of Active Compounds from Epimedium Grandiflorum Morr

Extracts of Epimedium grandiflorum Morr. were isolated and partitioned in order to identify the ERβ-selective active compounds in the extracts. Briefly, the dry leaves (1.8 Kg) of Epimedium grandiflorum Morr. were extracted with methanol:water (8:2, v:v) three times at room temperature (22.0 L and 8 h for each time). The combined methanol extracts were evaporated under vacuum to remove the organic solvent. The concentrated extracts were suspended in water (2.5 L) and sequentially partitioned with hexane, ethyl acetate, and n-butanol (2 L×3 times). The triplicate partitions were combined, concentrated, and assayed.

An ER-β specific assay was carried out essentially as described in Example 4 of U.S. patent application Ser. No. 11/298,957, filed Dec. 9, 2005, U.S. Pub. No. 2006/0134243 A1, which is incorporated herein by reference in its entirety. Briefly, a plasmid containing DNA encoding ER-β was co-transfected into cells along with a plasmid containing either TNF-RE-tk-Luc or ERE-tk-Luc as described in the Ser. No. 11/298,957 specification, for example in paragraph [0078]. The ER-β assay of the ethyl acetate extract obtained as described above showed good estrogenic activity; therefore, this fraction was subject to further column chromatography to purify active compounds. Silica gel (100 g), solid phase extraction C18 (10.0 g), Sephadex LH-10 (10.0 g) and Sephadex LH-20 media in open columns were utilized to separate and isolate active compounds for structural identification. Purified compounds were analyzed by LC-MS and NMR and data was compared to data acquired from authentic purchased standards.

NMR spectra were recorded using a Varian Mercury Plus 400 MHz in methanol-d4. The HPLC and UV spectrum were recorded using an Agilent Technologies 1200 Series HPLC system, equipped with a DAD detector, and using a Phenomenex Luna C18 column (150×2.1 mm, 3 μm).

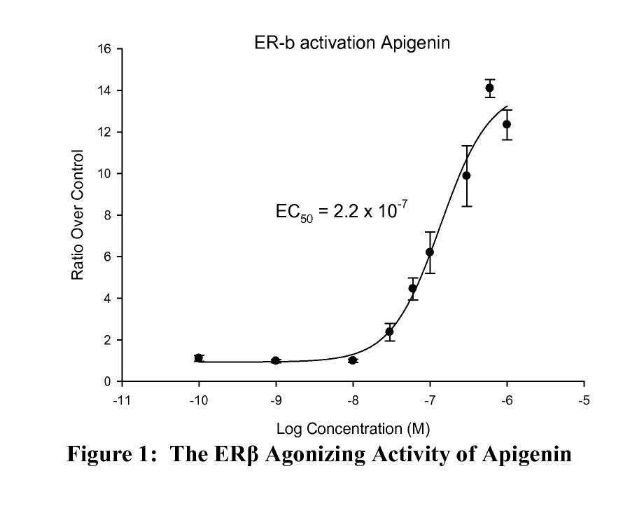

Seven compounds (Table 1-1) with ERβ-specific activity were identified from extracts of Epimedium grandiflorum Morr. All compounds were polyphenolic flavanoids with two compounds having sugar conjugates. Apigenin was the most active compound isolated and is a strong agonist of ERβ with an EC50 below 2.2×10−7 M (FIG. 1).

TABLE 1-1
Properties and structure of compounds identified from Herba Epimedium grandiflorum Morr.

| | | | Experiments | |
|---|---|---|---|---|
| Compound | Molecular | Molecular | supporting | |
| name | formula | weight | identification | Structure |
| Apigenin | C₁₅H₁₀O₅ | 270.24 | ¹H-NMR, LC-MS | 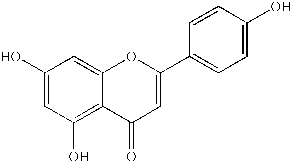 |
| Kaempferol | C₁₅H₁₀O₆ | 286.24 | ¹H-NMR, LC-MS | 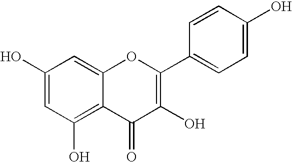 |
| Luteolin | C₁₅H₁₀O₆ | 286.24 | ¹H-NMR, LC-MS | 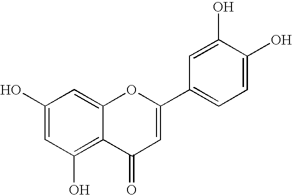 |
| Narigenin | C₁₅H₁₂O₅ | 272.25 | LC-MS | 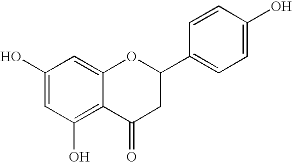 |
| Quercetin | C₁₅H₁₀O₇ | 302.24 | ¹H-NMR, LC-MS | 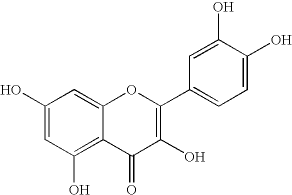 |
| Quercetrin | C₂₁H₂₀O₁₁ | 448.38 | ¹H-NMR, ¹³C-NMR, HSQC, HMBC, LC-MS | 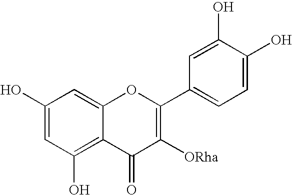 |
| Quercetin-3-O-β- glucopyranoside | C₂₁H₂₀O₁₂ | 464.38 | ¹H-NMR, ¹³C-NMR, HSQC, HMBC, LC-MS | 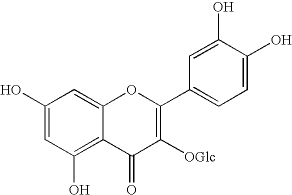 |

Each of the estrogenically active compounds isolated from extracts of E. grandiflorum Morr. (Table 1-1) differs structurally from each of the compounds identified as being estrogenically active by Yong et al., U.S. Pub. No. 2003/0170292 A1. For example, none of the isolated compounds from E. grandiflorum have isoprenyl moieties in their structures, whereas each of the compounds taught by Yong et al., has at least one isoprenyl group. These isoprenyl groups have very distinct chemical shifts when analyzed by NMR; and these chemical shifts were not observed in any of the ERβ-selective active fractions isolated from Epimedium grandiflorum Morr. In particular, Yong et al. identified Compound III as having ERβ-specific activity. This compound is not among the estrogenically active compounds listed in Table 1-1, and thus is not believed to be present in any appreciable amount in the extracts of E. grandiflorum Morr.

From the foregoing, it can be seen that the compounds identified as being estrogenically active components of E. grandiflorum Morr., which are recited in Table 1-1, above, are unexpectedly found in extracts of E. grandiflorum Morr. Such compounds possess ER-β-specific estrogenic activity. It is also apparent that these compounds are entirely different from those that have been identified in the prior art as being found in other species of Epimedii. Moreover, Compounds I and III taught by Yong et al. lacked any appreciable activity in the assays for estrogenic activity in the assays of this example.

### Example 2

### In Vivo Efficacy of Actives Derived from Epimedium Grandiflorum Morr

In order to demonstrate the safety and clinical activity of oral, a combination of active compounds isolated from Epimedium grandiflorum Morr. is studied in human patients with advanced breast cancer.

Eligible patients have histologically confirmed metastatic breast cancer and measurable disease. Patients do not receive any other chemotherapy, hormone therapy or herbal medicine during the trial. Patients receive 350 ml (equivalent to 0.00001-1 gram each of one, two, three, four, five, six, or all seven members of the group consisting of Apigenin, Luteolin, Kaempferol, Narigenin, Quercetin, Quercetrin, and Quercetin-3-O-β-glucopyranoside) of drug per day until disease progression, toxicity or personal preference caused them to discontinue. The primary endpoints are safety, toxicity and tumor response.

Patients are enrolled and receive drug. Mean age and mean number of prior treatments are recorded. Hematologic, and grade III or IV non-hematologic, adverse events (AEs), if any, are tracked and recorded. Patients who report grade I and II adverse events, such as nausea, diarrhea, headache, flatulence, vomiting, constipation, and fatigue, if any, are noted and recorded. Patients who are evaluable for response are evaluated and those with stable disease (SD) for >90 days and those with SD for >180 days are noted and recorded. Patients who have minor objective tumor regression are also noted and recorded.

Patients are enrolled at one or more suitable research centers and sign informed consent approved by local institutional review boards. Patients are excluded from the study for the following: extensive liver involvement (>50% of liver parenchyma), lymphangitic pulmonary involvement, central nervous system involvement or spinal cord compression not stabilized by therapy for >3 months, a history of multiple or severe food or medicine allergies and organ or marrow dysfunction as defined by creatinine>2.0 mg/dl, total bilirubin>1.7 mg/dl, white blood cell count<2,500 cells/μL and platelet count<75,000 mm³.

Safety monitoring is conducted on a continuous basis and patients are seen by a physician for examination at baseline at every Y weeks, wherein Y is a variable of from 1-4 weeks. Adverse events are graded using Common Toxicity Criteria version 2, assigned a category by organ system and coded in relation to study drug as remote, possible, probably or definitely related. Baseline tumor assessments are done within 14 days of initiation of study drug and every three months. Responses are assessed using RECIST criteria. Study drug is administered at every visit, and at this visit compliance and a review of dosages taken was performed. Study drug is provided as a liquid in a sealed and labeled aluminum packet containing a full daily dose that is administered in a split dose twice a day. Daily study drug is administered until the determination of tumor progression or dose limiting toxicity is encountered, or until the subject decides to voluntarily discontinue, in which case, the reason for discontinuation is obtained.

Results

Results of the above study are noted and evaluated based upon meeting the study endpoints.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical composition comprising one or more pharmaceutically acceptable excipients and one or more members of the group consisting of Apigenin, Luteolin, Kaempferol, Narigenin, Quercetin, Quercetrin, and Quercetin-3-O-β-glucopyranoside, wherein if the composition contains Apigenin, Luteolin or both, the composition also contains Kaempferol, Narigenin, Quercetin, Quercetrin, or Quercetin-3-O-β-glucopyranoside.

2. The pharmaceutical composition of claim 1, comprising two or more members of the group consisting of Apigenin, Luteolin, Kaempferol, Narigenin, Quercetin, Quercetrin, and Quercetin-3-O-β-glucopyranoside.

3. A method of selectively inducing apoptosis in a population of hyperproliferative cells in a mammal, comprising administering to the mammal a therapeutically effective amount of a composition of claim 1.

4. A method of selectively inducing apoptosis in a cancer in a mammal, comprising administering to the mammal a therapeutically effective amount of a composition of claim 1.

5. A method of treating cancer in a mammal, comprising administering to the mammal a therapeutically effective amount of a composition of claim 1.

6. A method of treating breast cancer, uterine cancer, ovarian cancer, cervical cancer, vulvar cancer, vaginal cancer, or endometrial cancer in a mammal, comprising administering to the mammal a therapeutically effective amount of a composition of claim 1.

7. A method of treating metastatic cancer originating in metastatic cancer originating in the breast, uterus, ovary, cervix, vulva, vagina, or endometrium in a mammal, comprising administering to the mammal a therapeutically effective amount of a composition of claim 1.
